(19) United States
(12) Patent Application Publication
(10) Pub. No.: US 2018/0237952 A1
(43) Pub. Date: Aug. 23, 2018

(54) KINK-RESISTANT ELECTROSPUN FIBER MOLDS AND METHODS OF MAKING THE SAME

(71) Applicant: NANOFIBER SOLUTIONS, INC., Wilmington, DE (US)

(72) Inventors: Jed JOHNSON, Columbus, OH (US); Tyler GROEHL, Columbus, OH (US)

(21) Appl. No.: 15/750,387

(22) PCT Filed: Aug. 5, 2016
(86) PCT No.: PCT/US16/45875

§ 371 Date: Feb. 5, 2018

Related U.S. Application Data
(60) Provisional application No. 62/201,269, filed on Aug. 5, 2015.

(51) Int. Cl. D01D 5/00; D04H 1/728; A61F 2/82; A61L 27/14

(52) U.S. Cl. CPC D01D 5/0084; D04H 1/728; A61F 2/07; A61L 27/14; A61L 2400/12; A61F 2/82

(57) ABSTRACT

A mandrel for forming a mold may comprise a rod having an outer surface, and a spiral component disposed around the outer surface of the rod, wherein the mandrel may be configured to receive an electrospun fiber. A method of making a kink-resistant electrospun fiber mold may comprise configuring such a mandrel to receive an electrospun fiber, applying a charge to one or more of the rod, the spiral component, and a polymer injection system, and depositing a polymer solution ejected from the polymer injection system onto the mandrel. A mold formed from such a method may comprise an inner wall extending axially, and an outer wall adjacent to the inner wall, having a plurality of axially adjacent, outwardly extending peaks separated by a plurality of valleys.

### CROSS-REFERENCE TO RELATED APPLICATIONS

This application claims priority to and benefit of U.S. Provisional Application Ser. No. 62/201,269 filed Aug. 5, 2015, entitled “Kink-Resistant Electrospun Fiber Molds and Methods of Making the Same,” the disclosure of which is incorporated herein by reference in its entirety.

### SUMMARY

In an embodiment, a mandrel for forming a mold may include a rod which has an outer surface, and a spiral component which is disposed around the outer surface of the rod. In an embodiment, the mandrel may be configured to receive one or more electrospun fibers.

In an embodiment, a method of making a kink-resistant electrospun fiber mold may include configuring a mandrel to receive a polymer fiber. In an embodiment, the mandrel may include a rod having an outer surface, and a spiral component disposed around the outer surface of the rod. In an embodiment, the method may further include applying a charge to the rod, the spiral component, a polymer injection system, or a combination thereof. In an embodiment, the method may further include depositing a polymer solution ejected from the polymer injection system onto the mandrel.

In an embodiment, a mold may comprise a structure formed from an electrospun fiber. In an embodiment, the structure may have an inner wall extending axially and an outer wall adjacent to the inner wall, the outer wall having one or more axially adjacent, outwardly extending peaks separated by one or more valleys.

### BRIEF DESCRIPTION OF THE DRAWINGS

FIG. 1A illustrates an embodiment of a mandrel in accordance with the present disclosure.

FIG. 1B illustrates an embodiment of a rod used in a mandrel in accordance with the present disclosure.

FIG. 1C illustrates an embodiment of a rod with a spiral component disposed around the outer surface of the rod, in accordance with the present disclosure.

FIG. 2A illustrates a standard cylinder mold with low kink resistance, as demonstrated by the bend and resulting occlusion shown therein.

FIG. 2B illustrates a spirally configured mold that may be flexed considerably without kinking, in accordance with the present disclosure.

FIG. 3 graphically illustrates the compliance of standard cylinder molds compared to that of spirally configured molds with the same diameter and wall thickness, in accordance with the present disclosure.

FIG. 4 illustrates a spirally configured mold implanted in vivo, in accordance with the present disclosure.

### DETAILED DESCRIPTION

Kink resistance is an important characteristic of any mold that may need to bend, coil, or flex for a given application. Kink resistance determines the degree to which a mold may be bent or formed before kinking. A kink within a mold may reduce, slow, occlude, or prevent the flow of a substance through the mold. Kink resistance may be particularly important for molds intended for use within biological organisms. In a subject's body, for example, the kinking of a luminal organ may reduce or prevent the flow of vital substances such as blood, gasses, or waste products, which could lead to serious illness, injury, or death. Molds comprising electrospun fibers, which may be used to replace such luminal organs, are often long, uniform cylinder structures with low kink resistance. This low kink resistance may be attributed to a lack of regions that are able to expand and contract.

The molds and associated methods disclosed herein may be used to form luminal electrospun fiber molds with thick and thin regions, or peaks and valleys, occurring periodically throughout the length of the mold. In some embodiments, the thin regions, or valleys, may have the ability to expand and contract, while the thick regions, or peaks, may maintain the mold's strength circumferentially. In some embodiments, the resulting mold may have a spiral configuration, allowing it to withstand high degrees of bending, flexing, and coiling without kinking.

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50% means in the range of 40% to 60%.

As used herein, the term “subject” includes, but is not limited to, humans, non-human vertebrates, and animals such as wild, domestic, and farm animals. In some embodiments, the term “subject” refers to mammals. In some embodiments, the term “subject” refers to humans.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. The resulting fiber molds or shapes may be used in many applications, including the repair or replacement of biological structures. In some embodiments, the resulting fiber mold may function as a scaffold for implantation into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h. In some embodiments, the flow rate may be, for example, about 0.5 mL/h, about 1 mL/h, about 1.5 mL/h, about 2 mL/h, about 2.5 mL/h, about 3 mL/h, about 3.5 mL/h, about 4 mL/h, about 4.5 mL/h, about 5 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.5 μm, about 1 μm, about 2 μm, about 5 μm, about 10 μm, about 20 μm, or ranges between any two of these values, including endpoints.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include synthetic or semi-synthetic polymers such as, without limitation, a polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polyvinyl alcohol (PVA), a polyvinyl acetate (PVAc), a polycaprolactone (PCL), a polylactic acid (PLA), a polyglycolic acid (PGA), a polyglycerol sebacic, a polydiol citrate, a polyhydroxy butyrate, a polyether amide, a polydiaxanone, and combinations or derivatives thereof. Alternative polymer solutions used for electrospinning may include natural polymers such as fibronectin, collagen, gelatin, hyaluronic acid, chitosan, or combinations thereof. It may be understood that polymer solutions may also include a combination of synthetic polymers and naturally occurring polymers in any combination or compositional ratio. In some non-limiting examples, the polymer solution may comprise a weight percent ratio of, for example, polyethylene terephthalate to polyurethane, from about 10% to about 90%. Non-limiting examples of such weight percent ratios may include about 10%, about 15%, about 20%, about 25%, about 30%, about 33%, about 35%, about 40%, about 45%, about 50%, about 55%, about 60%, about 65%, about 66%, about 70%, about 75%, about 80%, about 85%, about 90%, or any range between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, or any other materials to aid in non-invasive imaging, or any combination thereof. In some embodiments, the radiation opaque materials may include, for example, barium, tantalum, tungsten, iodine, or gadolinium. In some embodiments, the electrically conductive materials may include, for example, gold, silver, iron, or polyaniline.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternative fibers may be composed of polymers that may be absorbed or bio-degraded over time. Such fibers may act as an initial template or scaffold for the repair or replacement of organs and/or tissues. These organ or tissue templates or scaffolds may degrade in vivo once the tissues or organs have been replaced or repaired by natural structures and cells. It may be further understood that a polymer solution and its resulting electrospun fiber(s) may be composed of more than one type of polymer, and that each polymer therein may have a specific characteristic, such as stability or biodegradability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −3 kV, about −1 kV, about −0.01 kV, 0.01 kV, about 1 kV, about 5 kV, about 10 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Mandrel for Electrospinning Kink-Resistant Molds

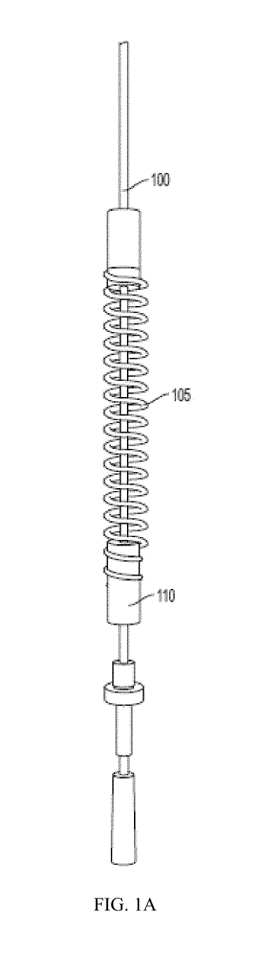

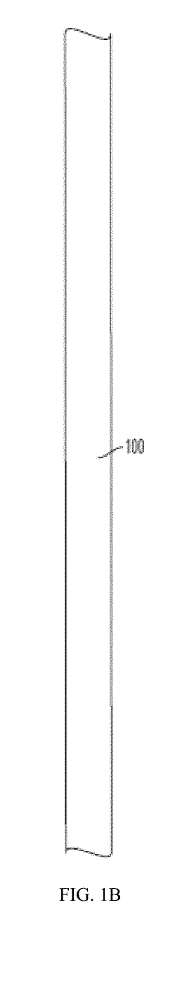

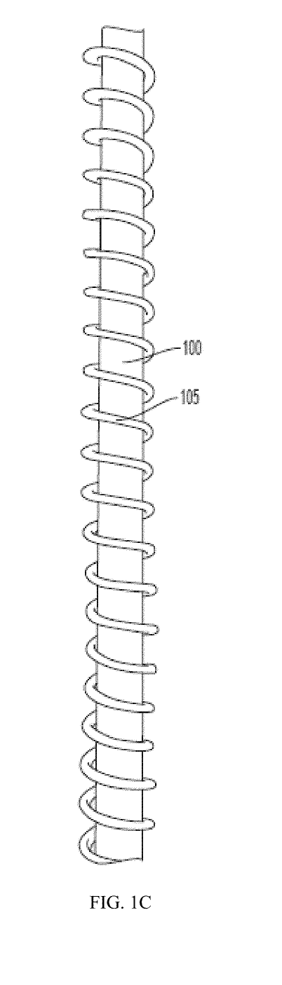

In some embodiments, the mandrel of an electrospinning system may be configured to form a kink-resistant fiber mold. In some embodiments, the mandrel may comprise a rod 100 having an outer surface, and a spiral component 105 disposed around the outer surface of the rod, as shown in FIGS. 1A, 1B, and 1C. In some embodiments, the spiral component 105 may be a spring. In some embodiments, the spiral component 105 may be another helical structure, such as a helical ceramic structure, a helical plastic structure, and the like. In some embodiments, the rod 100 and the spiral component 105 may be concentrically configured. In some embodiments, the mandrel may further comprise at least one spacing component 110 configured to separate the rod 100 and the spiral component 105.

In some embodiments, the spacing component 110 may comprise an insulating material. In some embodiments, the spacing component 110 may also support the orientation of the spiral component 105 about the rod 100.

In some embodiments, a mandrel comprising a rod and a spiral component, as described above, may attract polymer fibers proportionately to the spiral component and any spaces between the coils of the spiral component, resulting in an even distribution of the fibers and improved mechanical properties, including compliance and kink resistance. In some embodiments, the rod of a mandrel may be configured to receive a charge that may be different than the spiral component of the mandrel. In some embodiments, for example, the spiral component of the mandrel may be grounded. In an exemplary embodiment, the polymer injection system, or a portion thereof, may be positively charged, while the rod of the mandrel may be negatively charged, and the spiral component may be grounded. In some embodiments, the mandrel may be rotated during the electrospinning process, as described above, resulting in an even distribution of electrospun fibers over the mandrel. In some embodiments, the mandrel may be translated with respect to the polymer injection system. In some embodiments, a charged rod and a differently charged or grounded spiral component may allow the electrospun fibers to more uniformly cover the mandrel, resulting in a spirally configured electrospun mold with superior kink resistance.

In some embodiments, the rod of the mandrel may have an outer diameter from about 0.2 mm to about 80 mm. In some non-limiting examples, the outer diameter of the rod may be about 0.2 mm, about 0.5 mm, about 1 mm, about 2 mm, about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, or ranges between any two of these values, including endpoints.

In some embodiments, the spiral component of the mandrel may have an outer diameter from about 0.4 mm to about 110 mm. In some non-limiting examples, the outer diameter of the spiral component may be about 0.4 mm, about 0.6 mm, about 0.8 mm, about 1 mm, about 2 mm, about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, or ranges between any two of these values, including endpoints.

In some embodiments, the spiral component of the mandrel may have a wire gauge from about 40 to about 000 (3/0) (American wire gauge). In some non-limiting examples, the wire gauge of the spiral component may be about 40, about 39, about 38, about 37, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2, about 1, about 0 (1/0), about 00 (2/0), about 000 (3/0), or ranges between any two of these values, including endpoints.

In some embodiments, the spiral component of the mandrel may comprise from about 50 threads per inch to about 4 threads per inch. In some non-limiting examples, the spiral component of the mandrel may comprise about 50 threads per inch, about 45 threads per inch, about 40 threads per inch, about 35 threads per inch, about 30 threads per inch, about 25 threads per inch, about 20 threads per inch, about 15 threads per inch, about 10 threads per inch, about 8 threads per inch, about 7 threads per inch, about 6 threads per inch, about 5 threads per inch, about 4 threads per inch, or ranges between any two of these values, including endpoints.

In some embodiments, the rod and/or the spiral component of the mandrel may be coated with a non-stick material, such as, for example, aluminum foil, a stainless steel coating, polytetratluoroethylene, or a combination thereof, before the application of the electrospun fibers. The rod and/or the spiral component of the mandrel may be fabricated from aluminum, stainless steel, polytetrafitioroethylene, or a combination thereof to provide a non-stick surface on which the electrospun fibers may be deposited. In some embodiments, the rod and/or the spiral component of the mandrel may be coated with simulated cartilage or other supportive tissue. In some non-limiting examples, the rod and/or the spiral component of the mandrel may be configured to have a planar surface, a circular surface, an irregular surface, and a substantially cylindrical surface.

In some non-limiting examples, the mandrel comprising a rod and a spiral component may take the form of a bodily tissue or organ, or a portion thereof. In some non-limiting examples, the mandrel may be matched to a subject's specific anatomy. Non-limiting embodiments of such bodily tissues may include a trachea, one or more bronchi, an esophagus, an intestine, a bowel, a ureter, a urethra, a blood vessel, a nerve sheath (including the epineurium or perineurium), a tendon, a ligament, a portion of cartilage, a sphincter, a void, or any other tissue.

### Electrospun Kink-Resistant Molds

Electrospun fiber molds may be particularly useful for biological applications. Without wishing to be bound by theory, a synthetic scaffold which includes electrospun nanofibers may provide an ideal environment for biological cells, perhaps because a typical extracellular matrix configuration is also on the nanometer scale. It may be understood, therefore, that the molds and scaffolds described herein may be used in a wide variety of biological and surgical applications such as, for example, blood vessels, including peripheral blood vessels, intestines, and other gastrointestinal organs or portions thereof. The molds and scaffolds may be implanted without any cellular or biological materials, or they may be pre-conditioned to include such materials. In some non-limiting examples, the disclosed fiber molds may be seeded on both external and luminal surfaces with compatible cells that retain at least some ability to differentiate. In some embodiments, the cells may be autologous cells that may be isolated from the subject (e.g., from the subject's bone marrow) or allogeneic cells that may be isolated from a compatible donor. The seeding process may take place in a bioreactor (e.g., a rotating bioreactor) for a few weeks, days, or hours prior to implantation of the mold. Additionally, cells may be applied to the electrospun fibers immediately before implantation. In some embodiments, one or more growth factors may be added to the composition comprising the electrospun fibers immediately prior to implantation. The electrospun fibers incorporating such cells and/or additional chemical factors may then be transplanted or injected into the subject to repair or replace damaged tissue. The subject may be monitored following implantation or injection for signs of rejection or poor function. Any one or more of these procedures may be useful alone or in combination to assist in the preparation and/or transplantation of one or more tissues, or a portion of one or more tissues.

It may be appreciated that a variety of biological structures, tissues, and organs may be replaced or repaired by electrospun fiber molds. Some non-limiting examples of such structures may include a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, and portions thereof.

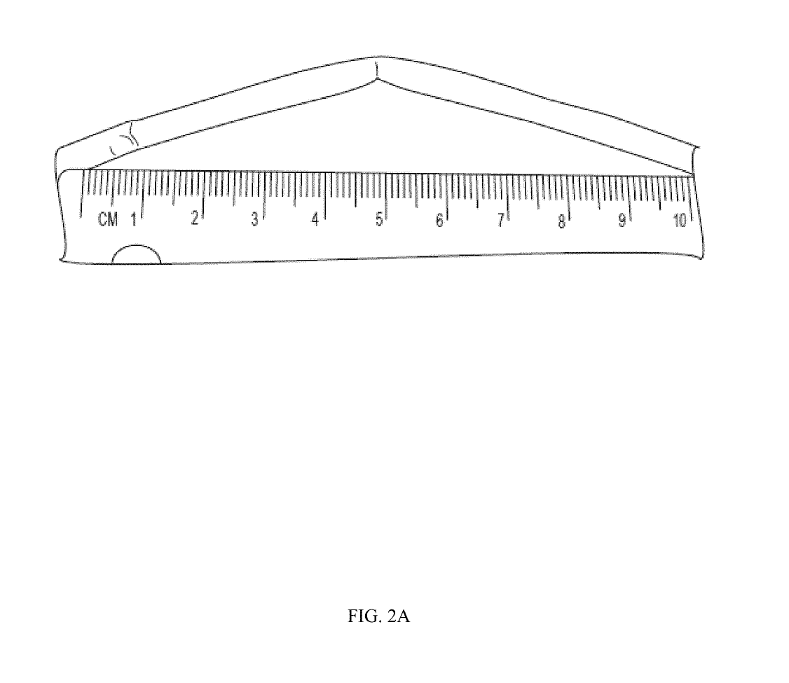

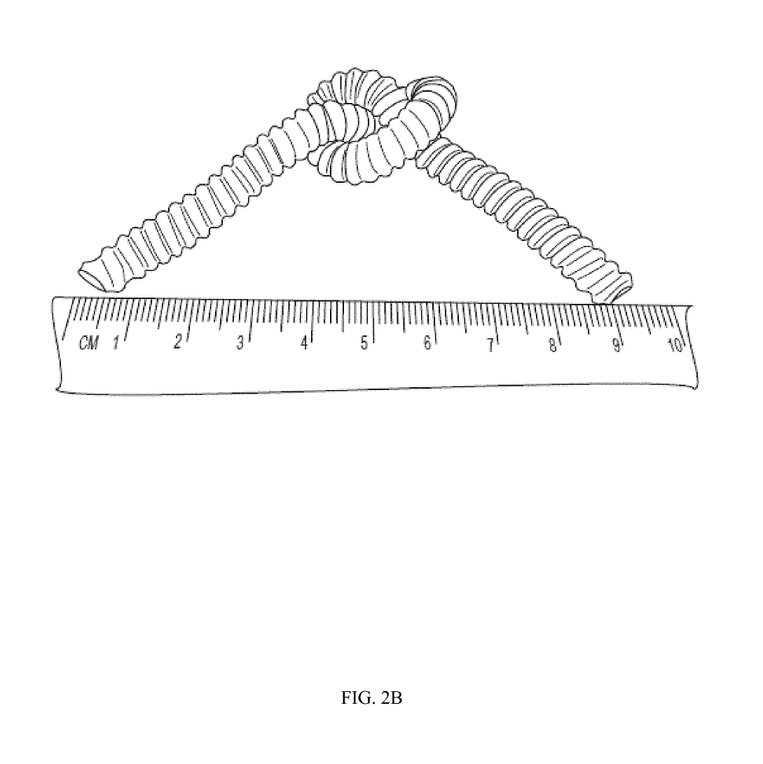

In some embodiments, the mold resulting from the use of the mandrel described above may comprise an inner wall extending axially, and an outer wall adjacent to the inner wall having a plurality of axially adjacent outwardly extending peaks separated by a plurality of valleys. The spacing of these peaks and valleys may be regular or irregular, and the minimum and maximum outer and inner diameters of these peaks and valleys may vary based on the mold's intended application. In some embodiments, the resulting mold with periodically spaced peaks and valleys may be more flexible than a uniformly shaped mold, and may be bent, curved, coiled, or otherwise deformed to a high degree without forming kinks or occlusions, as illustrated in FIGS. 2A and 2B. In some embodiments, the mold may have a spiral configuration. In some embodiments, the spiral configuration of an electrospun fiber mold may influence the flow of a substance, such as a fluid, through the mold. In some embodiments, the spiral configuration of the mold may encourage patency and discourage occlusions, even when the mold is bent, curved, coiled, or otherwise deformed.

In some embodiments, the mold may have one or more wall thicknesses from about 0.01 mm to about 10 mm. In an exemplary embodiment, the mold may have one or more wall thicknesses from about 0.1 mm to about 5 mm. In some non-limiting examples, the one or more wall thicknesses of the mold may be about 0.01 mm, about 1 mm, about 2 mm, about 3 mm, about 4 mm, about 5 mm, about 6 mm, about 7 mm, about 8 mm, about 9 mm, about 10 mm, or any ranges between any two of these values, including endpoints.

Conventional kink-resistant fiber molds may include rigid spiral components, such as metal springs, rigid plastic helices, and the like, which provide these molds with their purported kink resistance. It should be appreciated that the spirally configured electrospun fiber molds resulting from the use of the mandrel disclosed herein may not incorporate rigid spiral components; rather, their carefully controlled fiber compositions and orientations may allow them to have high compliance, favorable mechanical properties, and high kink resistance without the incorporation of such rigid spiral components.

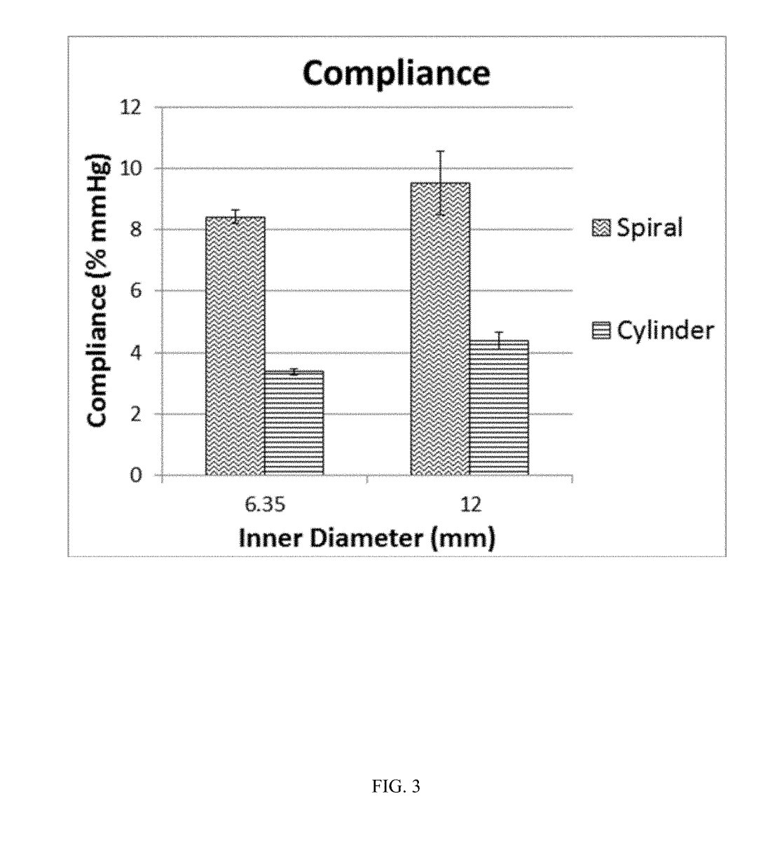

Spirally configured electrospun fiber molds in accordance with the present disclosure may have significantly increased compliance as compared to that of standard cylinder molds with the same diameter and wall thickness, as illustrated in FIG. 3.

### EXAMPLES

### Example 1

In one example, the compliance of a standard cylindrical mold was compared to that of a spirally configured mold in accordance with the present disclosure. For each graft, a 60 cc syringe was filled with water and placed in a syringe pump. The syringe pump was set to a constant flow rate of 5 mL/min. Surgical tubing was connected to the 60 mL syringe, and passed through a pressure transducer. The end of the surgical tubing was connected to a FR18 pediatric Foley catheter. A 2.5 cm long section of the vascular graft was positioned directly over the catheter balloon. The vascular graft section was centered in the field of view of a High Accuracy CCD Micrometer. Pressure and scaffold diameter readings were taken using Labview 2010 software and recorded four times per second. Testing was stopped at the point of failure of the graft, or when the pressure reached 30 psi, due to physical constraints of the catheter, tubing connections, and syringe pump. Compliance (C %) for this test was calculated using the compliance equation below, where PS is the systolic pressure, PD is the diastolic pressure, DS is the diameter at the systolic pressure, and DD is the diameter at the diastolic pressure.

$$C \% = \frac{\frac{{D}_{S} - {D}_{D}}{{D}_{D}}}{{P}_{S} - {P}_{D}} ⋆ {10}^{4} = \frac{\frac{{D}_{S}}{{D}_{D}} - \frac{{D}_{D}}{{D}_{D}}}{{P}_{S} - {P}_{D}} ⋆ {10}^{4} = \frac{\frac{{D}_{S}}{{D}_{D}} - 1}{{P}_{S} - {P}_{D}} ⋆ {10}^{4} .$$

FIG. 3 illustrates the results of this testing, and shows that the spirally configured electrospun fiber molds made in accordance with the present disclosure demonstrate significantly increased compliance as compared to that of standard cylinder molds with the same diameter and wall thickness.

### Example 2

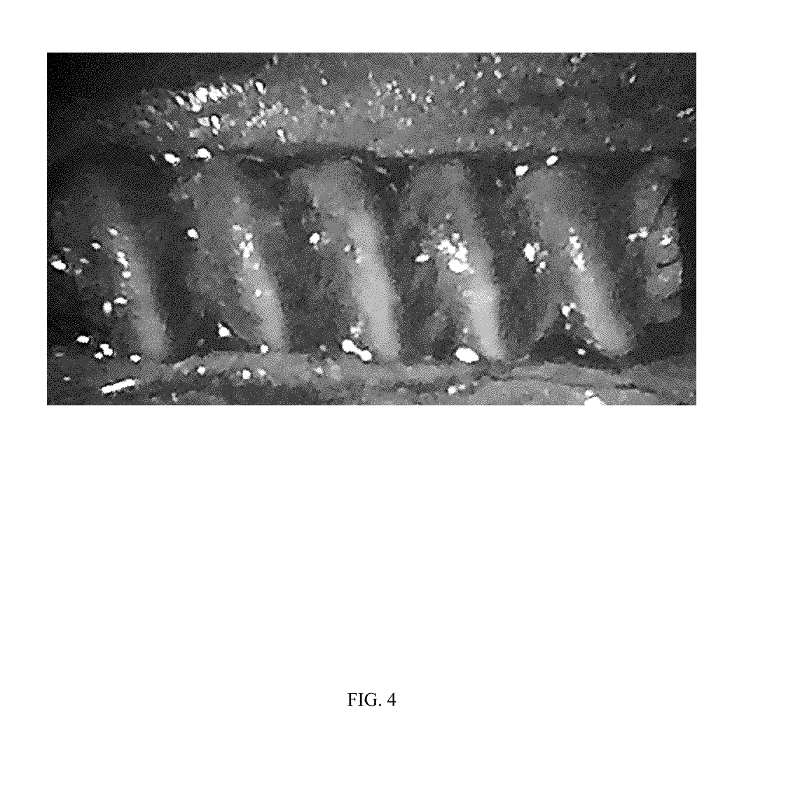

In another example, a spirally configured mold as described herein was implanted as an interposition infrarenal abdominal aortic (IAA) graft in a murine model. After 4 weeks in vivo, the graft appeared grossly patent, without evidence of aneurysmal dilation or stenosis. FIG. 4 illustrates the graft implanted in vivo in the murine model.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1.-28. (canceled)

29. A mandrel for forming a mold, the mandrel comprising:
a rod having an outer surface; and
a spiral component disposed around the outer surface of the rod;
wherein mandrel is configured to receive an electrospun fiber.

30. The mandrel of claim 29, wherein the spiral component is concentrically disposed around the rod.

31. The mandrel of claim 29, further comprising at least one spacing component configured to separate the rod and the spiral component, wherein the spacing component comprises an insulating material.

32. The mandrel of claim 29, wherein the rod is configured to receive a charge from about −0.01 kV to about −15 kV, and wherein the spiral component is configured to be grounded.

33. The mandrel of claim 29, wherein the rod comprises an outer diameter from about 0.2 mm to about 80 mm.

34. The mandrel of claim 29, wherein the spiral component comprises an outer diameter from about 0.4 mm to about 110 mm.

35. The mandrel of claim 29, wherein the spiral component comprises a wire gauge from about 40 to about 000 (3/0).

36. The mandrel of claim 29, wherein the spiral component comprises from about 50 threads per inch to about 4 threads per inch.

37. A method of making a kink-resistant electrospun fiber mold, the method comprising:
configuring a mandrel to receive a polymer fiber, the mandrel comprising a rod having an outer surface, and a spiral component disposed around the outer surface of the rod;
applying a charge to one or more of the rod, the spiral component, and a polymer injection system; and
depositing a polymer solution ejected from the polymer injection system onto the mandrel, wherein the polymer solution comprises a polymer and a solvent.

38. The method of claim 37, wherein applying a charge comprises applying a first charge from about −0.01 kV to about −15 kV, and applying a second charge from about 0.01 kV to about 30 kV.

39. The method of claim 37, further comprising grounding one or more of the rod, the spiral component, and the polymer injection system.

40. The method of claim 37, wherein applying a charge comprises applying a first charge to the rod from about −0.01 kV to about −15 kV, applying a second charge to the polymer injection system from about 0.01 kV to about 30 kV, and grounding the spiral component.

41. The method of claim 37, wherein the polymer is selected from the group consisting of a polyethylene terephthalate, a polyester, a polymethylmethacrylate, a polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone, a polylactic acid, a polyglycolic acid, a polyglycerol sebacic, a polydiol citrate, a polyhydroxy butyrate, a polyether amide, a polydiaxanone, derivatives thereof, and combinations thereof.

42. The method of claim 37, wherein the solvent is selected from the group consisting of acetone, dimethylformamide, dimethylsulfoxide, N-methylpyrrolidone, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, and combinations thereof.

43. The method of claim 37, further comprising mounting the mandrel onto a rotating motor having a rotational axis to align a longitudinal axis of the mandrel with the rotational axis of the motor and align the polymer injection system substantially perpendicular to rotational axis of the motor.

44. The method of claim 37, wherein the depositing comprises translating the mandrel substantially perpendicular with respect to the polymer injection system.

45. The method of claim 37, further comprising supporting the spiral component on the rod with a spacing component configured to separate the rod and the spiral component, wherein the spacing component comprises an insulating material.

46. A mold comprising:
a structure formed from an electrospun fiber, the structure having:
an inner wall extending axially; and
an outer wall adjacent to the inner wall having a plurality of axially adjacent, outwardly extending peaks separated by a plurality of valleys.

47. The mold of claim 46, wherein the plurality of peaks has a first outer diameter, and the plurality of valleys has a second outer diameter, and wherein the first outer diameter is larger than the second outer diameter.

48. The mold of claim 46, wherein the plurality of peaks and the plurality of valleys are disposed helically around the inner wall.
